(19) United States
(12) Patent Application Publication
(10) Pub. No.: US 2004/0260313 A1
(43) Pub. Date: Dec. 23, 2004

(54) Device and method for excising hair grafts

(76) Inventor: Per Gunnar Werner, Fagerstrand (NO)

Correspondence Address: CHRISTIAN D. ABEL, ONSAGERS AS, POSTBOKS 6963 ST. OLAVS PLASS, NORWAY, N-0130, NO

(21) Appl. No.: 10/204,949

(22) PCT Filed: Feb. 28, 2001
(86) PCT No.: PCT/NO01/00078

(30) Foreign Application Priority Data
Feb. 28, 2000 (NO) 20000980

(51) Int. Cl. A61B 17/50

(52) U.S. Cl. 606/131; 606/132; 606/133

(57) ABSTRACT

The invention relates to a device for reusing a strip of residue skin after drilling out grafts for the formation of additional grafts. The device comprises a support beam for supporting the strip of skin, equipped with elevated and recessed portions which are adapted to the shape of the strip of skin and in such a manner that two successive elevated or recessed portions define an area in the strip of skin which has to be cut up to form a graft. The invention also comprises a method wherein the strip of skin is placed on the support bar, a graft is excised by means of a cutting tool and it is transferred from the support bar to a magazine. The support bar, the magazine and the cutting tool are then moved relative to one another, thus making a new area of the strip of skin accessible to the cutting tool and a new receptacle in the magazine is provided in alignment with the said area.

The invention relates to a device and a method for reusing a strip of residual skin after drilling out grafts for the production of additional grafts. The invention permits the careful and rapid cutting up of grafts containing hair roots from donor skin which have been surgically removed from the nape of the patient's neck, —this excision taking place after a large number of grafts have already been drilled out of the donor skin by means of a hollow drill—, and subsequent insertion of the excised grafts into a magazine for immediate transfer to an implantation apparatus.

During the drill-out operation the donor skin is stretched in a tiltable frame and drilled through in closely spaced rows by a hollow drill which is adjusted in the direction of the hair follicles, thus preventing the hair follicles and hair roots from being cut. The hole spacing is preferably identical in the vertical and horizontal directions, and the spacing between the holes is preferably chosen so that the residue skin between the holes corresponds in size to the cylindrical, drilled-out grafts and thereby contains the same number of hair roots as these.

Experience shows that, when it is drilled according to the above-mentioned principle, the donor skin is strong enough to stick together properly during the drilling operation.

The donor skin's hair follicles are usually found to be at an angle of between 15° and 45° relative to a perpendicular on the skin surface. In order to be able to drill out the hair follicles and roots without damaging them, the frame has to be set at the same angle relative to the hollow drill. The holes produced by drilling will consequently be at the same angle relative to the skin plane.

When the drilling with the hollow drill is completed, slightly less than 50% of the hair roots have been removed from the donor skin. The just over 50% remaining are contained in the residue skin.

The residue skin's hair roots are for the most part located in the perforated area. This material contains approximately the same amount of usable material as the already drilled-out grafts.

The normal method to-day is for grafts to be excised from the whole of the donor skin and inserted in the skin of the patient's head manually, i.e. without any prior drill-out of the grafts. The excision is performed manually. A known, newer method involves approximately half of the grafts being first drilled out of the donor skin by means of a hollow drill. The residue skin, which contains approximately the same number of hair roots as the grafts which have been drilled out, is then cut up manually into grafts by means of simple manual tools such as scalpels and tweezers. This is a time-consuming task, and is generally carried out at normal room temperature. The long processing time and the high temperature during processing result in impairment of the hair roots.

It is crucial for the donor skin to be brought into a dormant state before and during the drilling and cutting process. This is achieved by keeping the skin cooled to just above freezing point from the time when it is removed from the skin of the neck until it is implanted in the skin of the head. When the graft is inserted in the skin of the head, the hair roots are revived due to warming and the supply of nourishment.

Experience shows that manually excised and implanted hair roots need a long time, usually a few months, to emerge from the dormant state and start to grow. The regrowth process for these grafts is also low as a result of the stresses to which they have been exposed. Mechanically excised grafts, which are cooled during processing and where this processing is performed rapidly, can provide regrowth in the course of two to three weeks, with a very high regrowth percentage.

U.S. Pat. No. 5,782,843 describes an apparatus for removal and implantation of grafts. The apparatus comprises a drill, a frame for stretching a piece of skin, an ejector pin, a magazine for receiving excised grafts and an implanting device. The apparatus therefore performs a mechanical excision and implantation. However, the apparatus is not suitable for cutting up the residue skin, since the skin which has to be drilled through must be kept taut and prevented from rotating with the drill. This can only be achieved if there is an ample amount of skin around the area which is being drilled out. The apparatus which is described in U.S. Pat. No. 5,782,843 is therefore only suitable for drilling out around 50% of the donor skin.

It should be mentioned at this point that donor skin which is surgically removed from the nape of a patient's neck has a substantial amount of hair roots, since the roots are very close together and are extremely viable. Nevertheless it is ethically untenable to waste skin, since the amount naturally is not unlimited.

The object of the invention is to meet the requirement for almost full utilisation of the valuable donor skin.

This object is achieved by means of a device according to the invention, which is characterized in that it comprises a support beam for supporting the strip of skin, where the support beam comprises elevated and recessed portions which are adapted to the shape of the strip of skin and in such a manner that two successive elevated or recessed portions define an area of the strip of skin which has to be cut up to form a graft. The invention also comprises a method for cutting tip grafts from the residue skin according to the invention. The method is characterized by: a) placing the strip of skin on a support beam, where the support beam comprises elevated and recessed portions which are adapted to the shape of the strip of skin and in such a manner that two successive elevated or recessed portions define an area corresponding to a graft on the strip of skin, b) providing a magazine with successively located receptacles for receiving grafts in the immediate vicinity of the support beam and in such a manner that a single receptacle is aligned with an area on the strip of skin, c) excising a graft by means of a cutting tool, d) transferring the graft from the support beam to the said receptacle by means of an ejector pin, e) moving the support beam, the magazine and the cutting tool relative to one another, thus making a new area of the strip of skin accessible to the cutting tool and providing a new receptacle in the magazine in alignment with the said area, f) repeating the steps c)-f).

The invention permits working with a strip of skin which is not stretched, but is only lying on a support beam.

The residue skin located outside the perforated area, i.e. skin located against the stretching beams and at the ends of the piece of skin, will still have to be cut manually into grafts. This border zone is so small that it can be cut up manually in the course of a relatively short time. In order to keep the graft's temperature down, use may be made of a refrigerated cabinet into which the operator concerned can put his hands.

Altogether, the drilling operation which is first performed on a piece of skin, the mechanised cutting up operation of the residue skin and the manual cutting up of the residue skin's border zones will cover practically 100% of the donor skin.

Since the drilling and excision are performed in the manner herein described and since these operations take place rapidly, with the result that the time between removal and implantation is short, the utilisation factor is very high. In addition, mechanically transplanted hair roots by means of the device and the method according to the invention will provide regrowth after two to three weeks.

In a preferred embodiment of the invention, the support beam's elevated portions are provided with cutting slots to permit the passage of a cutting tool, the depth of the slots preferably being adapted to the thickness of the strip of skin, their depth being sufficient to ensure complete excision of the grafts.

Also in a preferred embodiment the device comprises first transport devices for moving the support beam and the cutting tool relative to each other, with the result that the cutting slots in the support beam are sequentially accessible to the cutting tool. In a more preferred version, the first transport devices should be composed of a first rack attached to the support beam and a first pinion.

The device will further comprise a magazine for receiving the said grafts, the magazine comprising successively located receptacles for receiving and containing grafts one by one. In this connection the device will comprise second transport devices for moving the magazine and the support beam relative to each other and in such a manner that a single receptacle is made accessible for each area in the strip of skin and the subsequently excised graft. The second transport devices are preferably composed of a second rack attached to the magazine and a second pinion.

The spacing between two successive recessed portions or elevated portions in the support beam may be identical or different from the spacing between two successive receptacles.

Furthermore, an ejector pin will be employed for transferring the grafts from the support beam to the magazine. The support beam may be mounted between the ejector pin and the magazine or the magazine may be mounted between the ejector pin and the support beam.

The invention will now be described in more detail with reference to drawings in which:

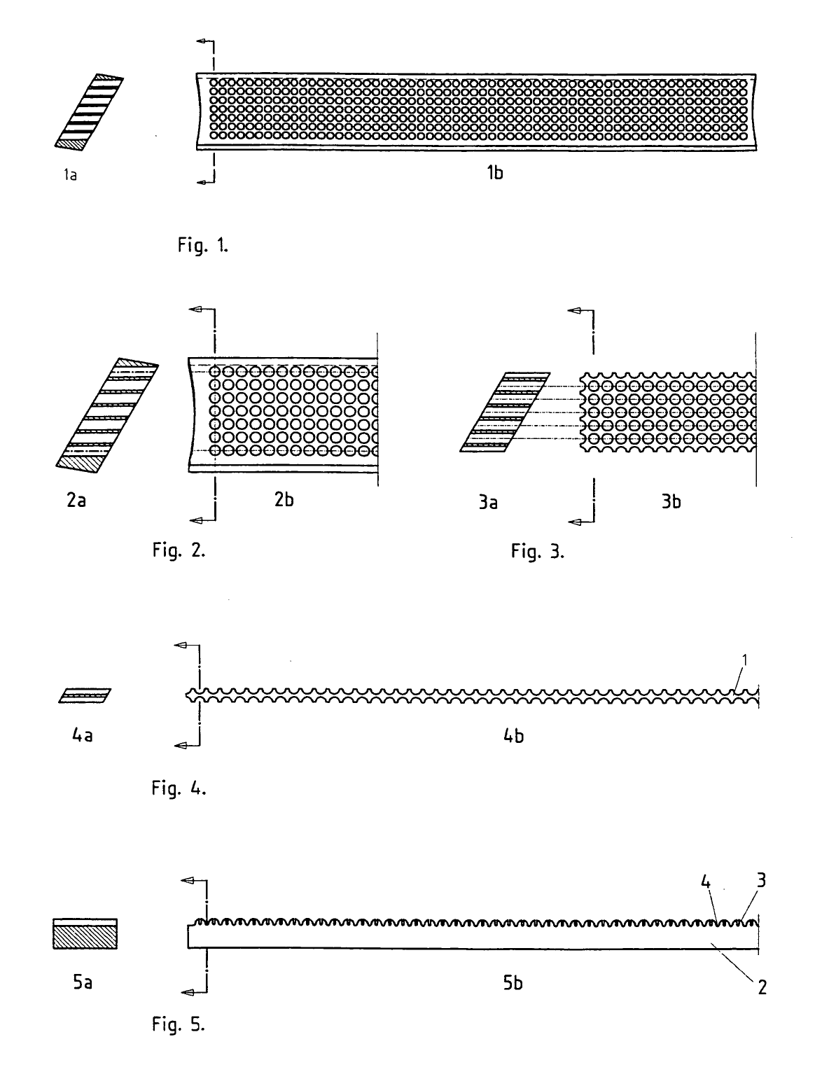

FIGS. 1a and 1b are an elevational view and a section illustrating drilled-out donor skin (residue skin),

FIGS. 2a and 2b are an elevational view and a section illustrating the cutting lines for the border,

FIGS. 3a and 3b are an elevational view and a section illustrating the cutting lines for the strips,

FIGS. 4a and 4b are an elevational view and a section illustrating a strip of the residue skin,

FIGS. 5a and 5b are an elevational view and a section illustrating the support beam according to the invention,

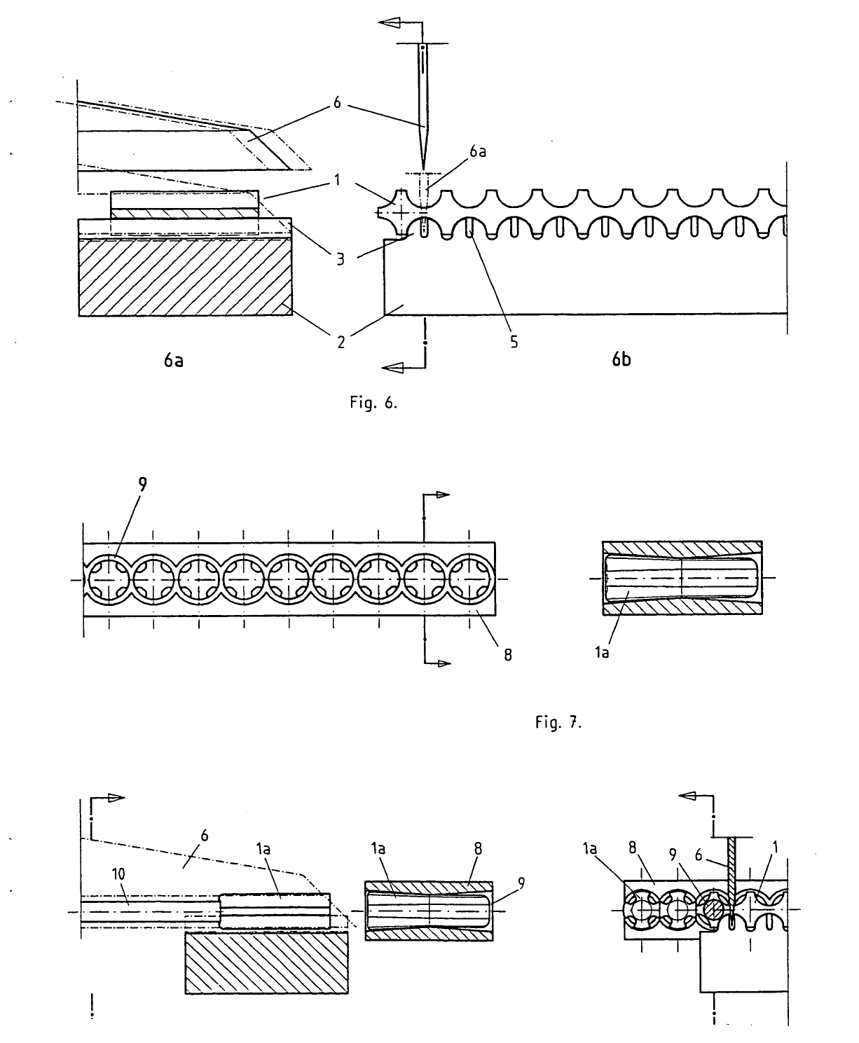

FIGS. 6a and 6b are an elevational view and a section illustrating the interaction between the support beam and the cutting tool,

FIGS. 7a and 7b illustrate a magazine filled with grafts,

FIGS. 8a, 8b and 8c illustrate the interaction between cutting tool, ejector pin, support beam, magazine and graft,

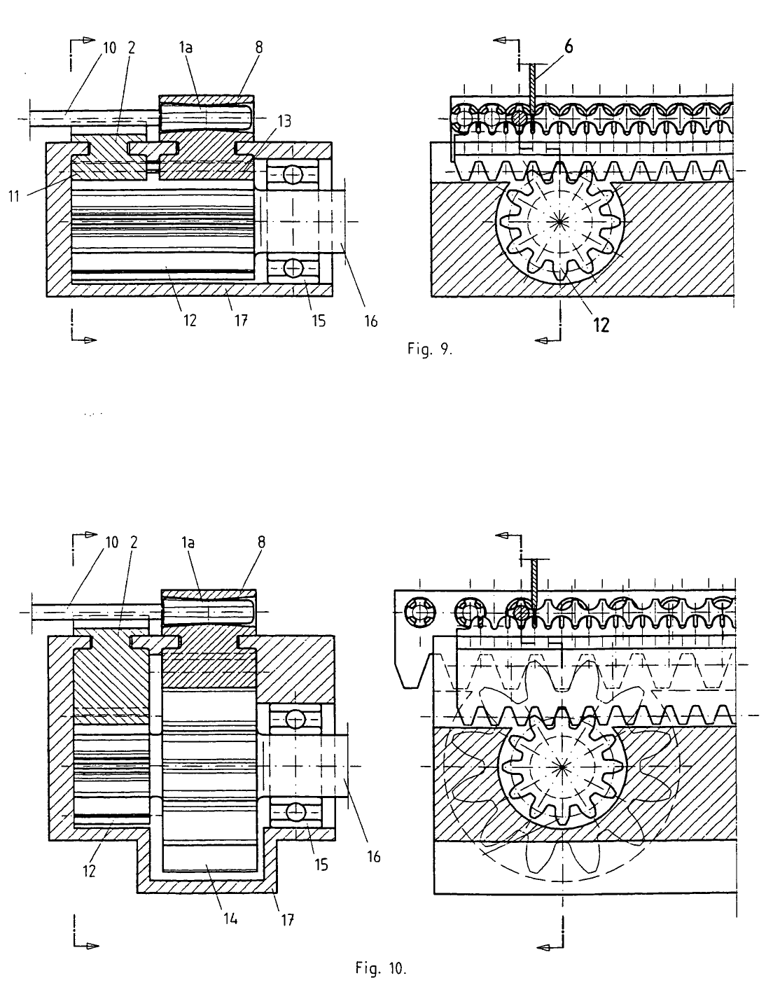

FIGS. 9a and 9b illustrate the transport devices for the support beam and the magazine and their interaction, and

FIGS. 10a and 10b illustrate a variant of FIGS. 9a and 9b.

FIG. 1 illustrates donor skin which is perforated by drilling with a hollow drill. In the figure, the donor skin's hair follicles and hair roots are tilted at 30° relative to a perpendicular on the skin's surface. The drillings are performed in the same direction. The figure illustrates the residue skin after the drilling operation.

FIG. 2 illustrates a section of the donor skin, where dot-and-dash lines indicate how the border round the perforated area is cut away after the drilling operation. This border area is cut up manually and used in the same way as the drilled-out and the mechanically excised grafts. Only a small area at the extreme edge of the skin border will be wasted. This area normally constitutes 2-5% of the skin piece. The utilisation factor will consequently be between 95 and 98%.

FIG. 3 illustrates a section of the residue skin within the perforated area after separation of the outer skin border. The dot-and-dash lines indicate the progress of the cuts when cutting up the residue skin into strips.

FIG. 4 illustrates an excised strip of the residue skin. The strip is preferably excised with a scalpel which is held at the same angle as the drillings. It is relatively easy to follow the drillings, and this cutting operation is done very quickly and accurately.

FIG. 5 is an elevational view and section illustrating a support beam 2 according to the invention. The support beam 2 is of a suitable metal, and comprises elevated portions 3 and recessed portions 4 for supporting and securing the strips of skin 1. The strip of skin 1 will be cut up into pieces which will form grafts. In a preferred embodiment of the invention the cutting up will take place in the area of each elevated portion 3 and each recessed portion 4, therefore defining a graft 1a. This will be explained in more detail later.

FIG. 6 illustrates the interaction between the support beam 2, the strip of skin 1 and the scalpel or cutting tool 6. The elevated portions 3 in the support beam are designed with narrow cutting slots 5, down into which the knife or scalpel 6 can go when cutting through the strip of skin 1. Thus it will be understood that the scalpel blade 6 cuts through the strip of skin 1 at the strip's thin portions (which in the illustrated embodiment correspond to the elevated portions 3). The slots 5 are of importance, since the skin fibres are extremely strong and tough. The knife edge 6a has to cut right through the skin 1 and deep down into the slots 5 in order to ensure complete excision. In order to further ensure complete excision of the strip of skin, the scalpel 6 should be moved backwards and forwards as indicated by dot-and-dash lines, while at the same time being moved downwards to the illustrated position near the bottom of the slot. When the scalpel 6 has reached the bottom position, a graft 1a is excised from the strip of skin 1, and it is lying ready to be transferred to a magazine for this purpose.

FIG. 7 illustrates a magazine 8 for receipt and short-term storage of excised grafts 1a and subsequent transport thereof to an apparatus for insertion of the grafts in the skin of the patient's head. The magazine has a number of borings or receptacles 9, each of which is arranged to receive and secure a graft. An embodiment of the said borings is illustrated in the figure, viz. a biconical boring. These borings guide the grafts in carefully, while holding the graft steady since they are slightly narrowed in the middle. The transfer of the graft 1a is carried out by means of an ejector pin 10, which is mounted in alignment with the graft's 1a centre line and which pushes it from the support beam 1 into one of the receptacles 9 in the magazine 8.

FIG. 8 illustrates the support beam 2 with the strip of skin 1 and an excised graft 1a, a scalpel blade 6, the magazine 8 for grafts and the ejector pin 10 which is arranged to push the graft 1a from the support beam 2 to the preferably cylindrical receptacles 9 in the magazine 8. The transport devices or the advancing mechanism for the support beam 2 and the magazine 8, the drive mechanism for the scalpel blade 6 and the ejector pin 10 and the surrounding details which keep the above-mentioned components in place are not shown in the figure. As illustrated in the figure the spacing between the recessed portions 4 in the support beam 2 are identical to the spacing between the individual receptacles 9 in the magazine 8. This corresponds to a preferred embodiment of the invention but is not the only possibility.

FIG. 9 illustrates the support beam 2, the graft 1a, the scalpel blade 6, the magazine 8 for grafts, and the ejector pin 10. The figure also illustrates the transport devices for the support beam 2 and the magazine 8. In a first embodiment of the invention the said devices comprise a first rack 11 which is attached to the support beam 2 and which is driven by a first pinion 12. The devices also comprise a second rack 13 which is attached to the magazine 8. In the embodiment of the invention which is illustrated in the figure (and which is the same as in FIG. 8) the spacing between the recessed portions 4 in the support bar 2 is identical to the spacing between the individual receptacles 9 in the magazine 8. In such a case it will be possible to provide a single common pinion 12 for both the racks 11 and 13. In other words, the support bar 2 and the magazine 8 will have rack teeth with the same module and number of teeth, with the result that they both move to the same extent when the pinion 12 is rotated. This is illustrated in the figure, where the pinion 12 is engaged with the support bar 2 and the magazine 8. The device further comprises a rotation bearing 15 for the pinion 12, the pinion's 12 shaft journal 16 and a housing 17 with linear bearing surfaces for the support bar 2 and the magazine 8 and a boring for the rotation bearing. The device further comprises a servo or stepping motor or a pawl device for stepwise advancement of the pinion 12 (not illustrated).

FIG. 10 illustrates a usual embodiment, where the spacing between the receptacles 9 in the magazine 8 is larger than the spacing between the recessed portions 4 in the support bar 2. The division in the support bar (and thus the spacing between the recessed portions 3) conforms to the desired drilling diameter for the grafts. The spacing between the receptacles 9 in the magazine 8, however, conforms to what is possible or desirable in practice in connection with the subsequent implantation operation. The latter spacing will normally be much larger than the former. A preferred solution to this problem is to employ two toothed wheels of different sizes (a first pinion 12 and a second pinion 14), mounted on the same drive shaft 16, where the smaller of the toothed wheels 12 drives the support bar 2 and the larger toothed wheel 14 drives the magazine 8. At the location where the cutting takes place the graft 1amust be aligned with the receptacles 9 in the magazine 8. Between the support bar 2 and the magazine 8 it may be necessary to place a funnel-shaped guide which guides the graft 1a into the receptacle 9. This funnel is not illustrated in the figure.

1. A device for the mechanical production of grafts containing hair roots from perforated donor skin, characterized in that the device comprises a support beam (2) for supporting the strip of skin (1), the support beam comprising elevated and recessed portions (3,4) which are adapted to the shape of the strip of skin (1) and such that two successive elevated or recessed portions (3 or 4) define an area in the strip of skin (1) which has to be cut up to form a graft (1a).

2. A device according to claim 1,
characterized in that the elevated portions (3) are provided with cutting slots (5) to permit the passage of a cutting tool (6), the depth of the slots (5) preferably being adapted to the thickness of the strip of skin (1) in such a manner that it is sufficiently deep to ensure complete excision of the grafts (1a).

3. A device according to claim 2,
characterized in that it comprises first transport devices for moving the support beam (2) and the cutting tool (6) relative to each other so that the cutting slots (5) in the support beam (2) are sequentially accessible to the cutting tool (6).

4. A device according to claim 3,
characterized in that the first transport devices are composed of a first rack (11) attached to the support beam (2) and a first pinion (12).

5. A device according to one of the preceding claims,
characterized in that it further comprises a magazine (8) for receiving the said grafts (1a), the magazine (8) comprising successively located receptacles (9) for receiving and containing grafts (1a) one by one.

6. A device according to claim 5,
characterized in that the spacing between two successive recessed portions (4) or elevated portions (3) in the support beam (2) is different from the spacing between two successive receptacles (9), and that the device further comprises second transport devices for moving the magazine and the support beam (2) relative to each other and in such a manner that a single receptacle (9) is made accessible for each area in the strip of skin (1) and the subsequently excised graft (1a).

7. A device according to claim 6,
characterized in that the second transport devices are composed of a second rack (13) attached to the magazine (8) and a second pinion.

8. A device according to one of claims 5-7,
characterized in that an ejector pin (10) is employed for transferring the grafts (1a) from the support beam (2) to the magazine (8), and the support beam (2) is mounted between the ejector pin (10) and the magazine (8).

9. A device according to one of claims 5-8,
characterized in that an ejector pin (10) is employed for transferring the grafts (1a) from the support beam (2) to the magazine (8), and the magazine (8) is mounted between the ejector pin (10) and the support beam (2).

10. A method for reusing a strip of residue skin after drilling out grafts for the formation of additional grafts,
characterized by
a) placing the strip of skin (1) on a support beam (2), where the support beam (2) comprises elevated and recessed portions (3, 4) which are adapted to the shape of the strip of skin (1) and in such a manner that two successive elevated or recessed portions (3 or 4) define an area corresponding to a graft (1a) on the strip of skin (1),
b)providing a magazine (8) with successively located receptacles (9) for receiving grafts (1a) in the immediate vicinity of the support beam (2) and in such a manner that a single receptacle (9) is aligned with an area on the strip of skin (1),
c) excising a graft (1a) by means of a cutting tool (6),
d) transferring the graft (1a) from the support beam (2) to the said receptacle (9) by means of an ejector pin (10),
e) moving the support beam (2), the magazine (8) and the cutting tool (6) relative to one another, thus making a new area of the strip of skin (1) accessible to the cutting tool (6) and a new receptacle (9) in the magazine (8) is provided in alignment with the said area,
f) repeating the steps c)-f).
